(19) United States
(12) Patent Application Publication
(10) Pub. No.: US 2010/0301877 A1
(43) Pub. Date: Dec. 2, 2010

(54) FLEXIBLE SHEET SENSOR INSERTED IN TUBE

(76) Inventors: Neil D Paterson, South Australia (AU); Alf Cousins, South Australia (AU)

Correspondence Address: ARTHUR G. SCHAIER;CARMODY & TORRANCE LLP, 50 LEAVENWORTH STREET, P.O. BOX 1110, WATERBURY, CT, 06721, US

(21) Appl. No.: 12/312,238

(22) PCT Filed: Nov. 2, 2007
(86) PCT No.: PCT/AU2007/001671

§ 371 Date: May 20, 2010

(30) Foreign Application Priority Data
Nov. 3, 2006 (AU) 2006906136

(51) Int. Cl. G01R 27/26; G01N 33/24

(52) U.S. Cl. 324/664

(57) ABSTRACT

A sensing unit (100) for sensing a parameter of a medium surrounding a tubular member (110) is disclosed. The sensing unit (100) includes an electronics module (102), a flexible sheet (104) and a shaped member (108). The flexible sheet (104) includes one or more sensing elements (106) that interface with the electronics module (102) to form a sensing circuit for sensing the parameter of the medium. The flexible sheet (104) is formable around the outside of the shaped member (108) to provide an insert (200) for insertion into the tubular member (110). After insertion into the tubular member (110) the insert (200) bears against an inner wall (300) of the tubular member (110) so that the shape of the one or more sensing elements (106) adapts to the shape of the inner wall (300) of the tubular member (110).

This application claims priority from Australian Provisional Patent Application No. 2006906136 filed on 3 Nov. 2006, the contents of which are to be taken as incorporated herein by this reference.

### FIELD OF THE INVENTION

The present invention relates to a sensing unit and to a sensor assembly incorporating such a sensing unit. The sensing unit has particular but not exclusive application in the field of soil moisture and/or salinity measurement.

### BACKGROUND OF THE INVENTION

Various forms of sensors, such as soil moisture sensing probes, have been developed to enable measurement of soil parameters such as soil moisture and/or soil salinity levels. One such soil moisture probe includes a plastic access tube that is installed in the soil of a monitored site. A sensing unit is suspended inside the access tube and measures parameters to infer soil moisture and/or soil salinity levels of the soil surrounding the access tube. Sensors of this type typically rely on measuring a frequency change of a radio frequency signal in an oscillator circuit having a capacitive element (for example a pair of electrodes) that projects an electric field into a “sphere of influence” of the soil being measured.

In conventional soil moisture sensing probes, in order to enable the sensing unit to be located within the access tube some clearance is provided between the periphery of the sensing unit and the inner wall of the access tube. This clearance typically results in an air gap being formed between the inner wall of the access tube and an outer side surface of the sensing unit.

One difficulty with sensors of this type is that the resulting parameter measurements will only be accurate if the thickness of the wall of the access tube and the width of any air gap between the sensing unit and the access tube wall are properly taken into consideration when calibrating the probe. Such sensors are generally calibrated to account for access tube wall thickness by using a sample of the access tube being used in the probe. Unfortunately, such calibration is not particularly accurate because it cannot account for any variations in wall thickness along the length of the access tube actually used for the sensor in the installation. Similarly, it is difficult to calibrate the sensor to account for actual variations in the air gap along the length of the access tube.

The present invention seeks to provide an improved form of sensing unit or sensor assembly which addresses at least some of the above mentioned difficulties.

### SUMMARY OF THE INVENTION

The present invention provides a sensing unit for sensing a parameter of a medium surrounding a tubular member, the sensing unit including: an electronics module; a flexible sheet including one or more sensing elements interfacing with the electronics module to form a sensing circuit for sensing the parameter of the medium; and a shaped member; wherein the flexible sheet is formable around the outside of the shaped member to provide an insert for insertion into the tubular member, and wherein after insertion into the tubular member the insert bears against an inner wall of the tubular member so that the shape of the one or more sensing elements adapts to the shape of the inner wall of the tubular member.

The electronics module may include a processing unit and associated circuitry (such as bypass capacitors, pull-up resistors and the like) for processing a sensed signal generated by the sensing circuit. The processing module may include, for example, a programmed controller, such as a micro-controller including on-board memory containing program instructions in the form of application code. One suitable processing module is an ATMEGA168 controller including 16 Kbyte on-board memory.

The circuitry of the electronics module, including the processing module and the associated circuitry, may be mounted on a rigid printed circuit board (PCB) or a flexible PCB. The actual configuration of the electronics module, at least in terms of its hardware and software design, will vary according to the characteristics of the sensing elements and the parameter being sensed. Typically, the sensing elements are capacitive elements and the parameter of the medium being sensed is moisture. Thus, in one embodiment, the sensing circuit generates a sensed signal having a signal parameter value attributable to a moisture content parameter of a soil medium. In such an embodiment, the processing module processes the signal parameter value to provide, at an output, a scaled data value indicative of a value of moisture content. In such an embodiment, the sensing circuit will typically include an oscillator circuit that itself includes a paired arrangement of sensing elements provided with the flexible sheet. The paired sensing elements provide a capacitive element having a value of capacitive reactance attributable to the dielectric constant of the soil medium and thus attributable to the soil moisture content.

The oscillator circuit may include a balanced very high frequency (VHF) voltage controlled oscillator tuned via a differential capacitance circuit that includes the capacitive element. In an embodiment, the oscillator has a resonant frequency that varies over the range of about 90.00 MHz to 165.00 MHz.

The flexible sheet may be formed as a flexible printed circuit board (PCB) that is arranged in use for connection to, or that is integrally formed with, the electronics module. In this respect, in an embodiment in which the electronics module includes a flexible PCB, that flexible PCB may be integrally formed with the same PCB as the electronics module. However, it is not essential that the flexible sheet be integrally formed with, or even directly mechanically connected to, the electronics module. Indeed, in one embodiment the electronics module is located within the tubular member but is separate from the flexible sheet. However, in such an embodiment, the one or more sensing elements of the flexible sheet will be electrically connected to the electronics module by way of a suitable connection scheme, such as a cable, to form the sensing circuit.

The one or more sensing elements are preferably made from flexible conductive (for example, copper) strips that are formed integrally with the flexible PCB of the flexible sheet. In other words, the flexible PCB of the flexible sheet and the flexible conductive strips may provide a one-piece construction.

In an embodiment for sensing the moisture content of a soil medium, the conductive strips may be arranged in pairs to form respective plates of a capacitive element of the sensing circuit. Accordingly, in such an embodiment, each of one or more pairs of conductive strips provides a respective capacitor.

In an embodiment, the configuration of the sensing elements, and thus of the plates of each capacitor, at least in terms of their longitudinal cross section (that is, a cross section perpendicular to the longitudinal axis of the shaped member), is established after the insert has been inserted into the tubular member. Once inserted, each flexible conductive strip forms a band extending about the longitudinal axis of the shaped member. Typically, the band circumscribes the longitudinal axis.

The flexible sheet may be rectangular in shape and include a secured side and three free sides. Typically the secured side will have substantially the same length as the length of the shaped member, but other configurations may be used. The width of the rectangular sheet will typically be substantially the same as, or greater than, the inner circumference of the tubular member so that when the insert is inserted into the tubular member substantially the entire outer side surface of the flexible sheet makes contact with the inner wall of the tubular member.

In an embodiment, the flexible sheet includes plural sensing elements arranged as mutually parallel conductive strips spanning from the secured side of the flexible sheet towards the free side opposite the secured side. In an embodiment in which the conductive strips form plates of a capacitor, such an arrangement permits, in use, the conductive strips to establish, for each capacitor, a sensing zone that surrounds the outer wall of the tubular member.

The secured side of the flexible sheet may be attached to the electronics module, or the shaped member, to thereby secure that side relative to the shaped member. In either case, forming the insert entails wrapping the flexible sheet from the secured side and around the outside of the shaped member so that the flexible sheet has an inner surface that wraps around the outside of the shaped member to thereby form an insert having a suitable configuration for inserting into the tubular member. For example, in one embodiment in which the tubular member is a hollow cylinder, the insert will adopt a generally cylindrical configuration.

The shaped member may be an elongate channel manufactured from a material such as, for example, polyurethane. However, it is not essential that the shaped member be a channel as other forms may also be suitable. Indeed, in one embodiment the shaped member is a solid shape, such as a cylinder, manufactured from a resilient material.

The shaped member may be a one-piece construction, or it may include sections that are assembled to form the shaped member. Irrespective of the actual configuration of the shaped member, the shaped member may include one or more resilient surfaces for contacting areas of an inner surface of the flexible sheet when the flexible sheet is formed around the outside of the shaped member. In such an embodiment, the resultant insert is deformable from an “initial configuration” to an “insertion” configuration in which its dimension is reduced for insertion into the tubular member. In other words, in accordance with one embodiment of the present invention, the insert is configured so that when it is located in the tubular member, the outer side surface of the flexible sheet is biased against the inner wall of the tubular member. In an embodiment, the biasing of the outer side surface of the flexible sheet against the inner wall of the tubular member is provided by the resilient combination of the flexible sheet and the shaped member.

The insert may adopt a generally tubular configuration that is sufficiently deformable to enable the insert to be resiliently deformed from the “initial” configuration to the “insertion” configuration.

The resilient surface may be a single surface that extends about a longitudinal axis of the shaped member. Alternatively, in an embodiment that includes plural resilient surfaces, each surface may be arranged to provide guides for shaping the flexible sheet when forming the insert. The guides may include outwardly projecting flanges, extending lengthwise along the shaped member, and spaced apart with respect to one another apart to provide a spacing therebetween. In such an embodiment, after forming the insert, areas of the flexible sheet spanning the spacings will be manipulable to an extent by applying a radially inward force thereto so as to reduce a dimension between the respective area of the flexible sheet and the longitudinal axis of the shaped member. In such an embodiment, the configuration of the flanges, combined with the resilient nature of the flexible sheet, biases the outer side surface of the flexible sheet against the inner wall of the tubular member after insertion of the insert into the tubular member so that the shape of the one or more sensing elements adapts to the shape of the inner wall of the tubular member.

As will be appreciated, the arrangement of the resilient surfaces may vary according to the desired configuration of the insert. For example, in an embodiment in which the insert is to adopt a generally tubular configuration, the resilient surfaces may include arcuate flanges that are shaped and arranged to provide a guide that defines an inner dimension, such as an inner diameter, of the inner surface of the sensing unit after formation of the insert.

Once inserted within the tubular member the insert will endeavour to return to its “initial” configuration. Subject to the tubular member having an appropriate inner dimension, the resilient nature of the insert will thus contribute to the outer side surface of the flexible sheet being biased against the inner wall of the tubular member so that the shape of the one or more sensing elements adapts to the shape of the inner wall of the tubular member, to thereby minimise any air gap located therebetween.

The shaped member may include a support arrangement for supporting the electronics module. In one embodiment, the shaped member includes a planar section for receiving and supporting the electronics module. The planar section may include a web located between and connected to flanges of the shaped member. On one surface of the web, oppositely arranged clamping members may be provided for securing the electronics module. In one embodiment, the web is resilient and sufficiently flexible to permit the clamping members to be manipulated with respect to each other for clamping and releasing the electronics module.

It will be appreciated by those skilled in the art that the insert may be installed within a tubular member previously installed in a monitoring area. Alternatively, such an insert may be installed in a new tubular member prior to mounting in a monitored area. Accordingly, the present invention also provides an insert for inserting into a tubular member of a sensing unit for sensing a parameter of a medium, the insert including:

a flexible sheet including one or more sensing elements for interfacing with an electronics module to form a sensing circuit for sensing the parameter of the medium surrounding a tubular member; and

a shaped member;

wherein the insert is formed by forming the flexible sheet around the outside of the shaped member.

Typically, the tubular member is formed from a length of PVC piping. Top and bottom end caps may be secured to the tubular member so that the sensing unit is sealed to prevent access by contaminants. In an assembled form, the present invention also provides a sensor for sensing moisture content of a soil medium, the sensor including:

a tubular member;

an electronics module;

a flexible sheet including one or more sensing elements interfacing with the electronics module to form a sensing circuit for sensing the parameter of the soil medium surrounding the tubular member; and

a shaped member;

wherein the flexible sheet is formed around the outside of the shaped member to provide an insert which is located within the tubular member and wherein the one or more sensing elements substantially adopts the shape of an inner wall of the tubular member.

Embodiments of the present invention are expected to find application in numerous areas of application. For example, a sensor including a sensing unit in accordance with an embodiment of the present invention may be used in irrigation applications such as agricultural irrigation, viticultural irrigation, horticultural irrigation, domestic and commercial garden irrigation, urban open space irrigation, turf-grass irrigation, and sports playing field (such as golf course irrigation). Of course, it will be appreciated that the present invention is not limited to irrigation applications. Indeed, the present invention could also find application in site remediation monitoring, mining site dewatering control, sewerage and drainage control, construction site environmental monitoring, industrial, commercial and process plant/process/air handling monitoring, domestic, commercial and industrial building footings, geotechnical monitoring and control, environmental monitoring, and underground tunnel geotechnical monitoring.

### BRIEF DESCRIPTION OF THE DRAWINGS

Embodiments of the invention will now be described, by way of example only, with reference to the accompanying drawings in which:

FIG. 1 is an exploded view of a sensor in accordance with an embodiment of the present invention;

FIG. 2A is a perspective view of the sensing unit incorporated in the sensor in FIG. 1, prior to forming an insert for inserting into the tubular member;

FIG. 2B is a perspective view of the sensing unit shown in FIG. 2A during forming the insert for inserting into the tubular member;

FIG. 2C is another perspective view of the sensing unit shown in FIG. 2A, after forming an insert for inserting into the tubular member in FIG. 1;

FIG. 3A is a perspective view of the sensor shown in FIG. 1, with a section shown in cut-away view;

FIG. 3B is a close-up view of the section shown in cut-away in FIG. 3A;

FIG. 4 is a cross sectional view of the insert illustrated in FIG. 2C; and

FIG. 5 is a cross sectional view of a tubular member suitable for use with an embodiment of the sensing unit illustrated in FIG. 1;

FIG. 6 is a cross sectional view of the insert illustrated in FIG. 2C shown inserted within the tubular member shown in FIG. 1; and

FIG. 7 is a perspective view of a sensor shown in FIG. 1.

### DETAILED DESCRIPTION OF AN EMBODIMENT

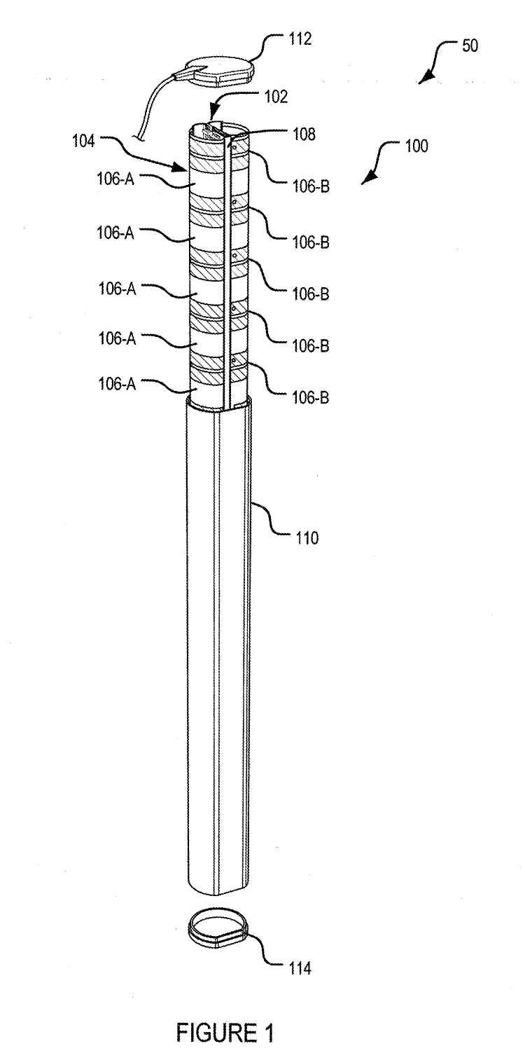

FIG. 1 depicts an exploded view of a sensor 50 including a sensing unit 100 in accordance with an embodiment of the present invention. The sensing unit 100 includes an electronics module 102, a flexible sheet 104, and a shaped member 108. As shown, the sensor 50 also includes a tubular member 110, top 112 and bottom 114 end caps for securing to the tubular member 110 so as to prevent entry of moisture or other contaminants thereinto which may damage the sensing unit 100.

In the embodiment illustrated, the sensor 50, and thus the sensing unit 100, is for sensing a parameter of a soil medium surrounding the tubular member 110. In the present case, the sensed parameter is the soil moisture content of the soil medium. Thus, the illustrated sensor 50 is arranged to be installed in the soil of a monitoring site and the sensing unit 100 is designed to sense the moisture content of the soil medium surrounding the tubular member 110.

The electronics module 102 includes a processing unit (not shown) and associated circuitry (not shown) for processing a sensed signal generated by a sensing circuit. In the present case, the processing unit includes an ATMEGA168 controller including 16Kbyte on-board memory containing program instructions in the form of application code. In the illustrated embodiment the circuitry of the electronics module 102 is mounted on a flexible PCB.

The flexible sheet 104 includes pairs of sensing elements 106-A, 106-B (shown as solid areas spaced apart by areas represented using hatched lines). The function of the sensing elements 106-A, 106-B will be described in more detail later.

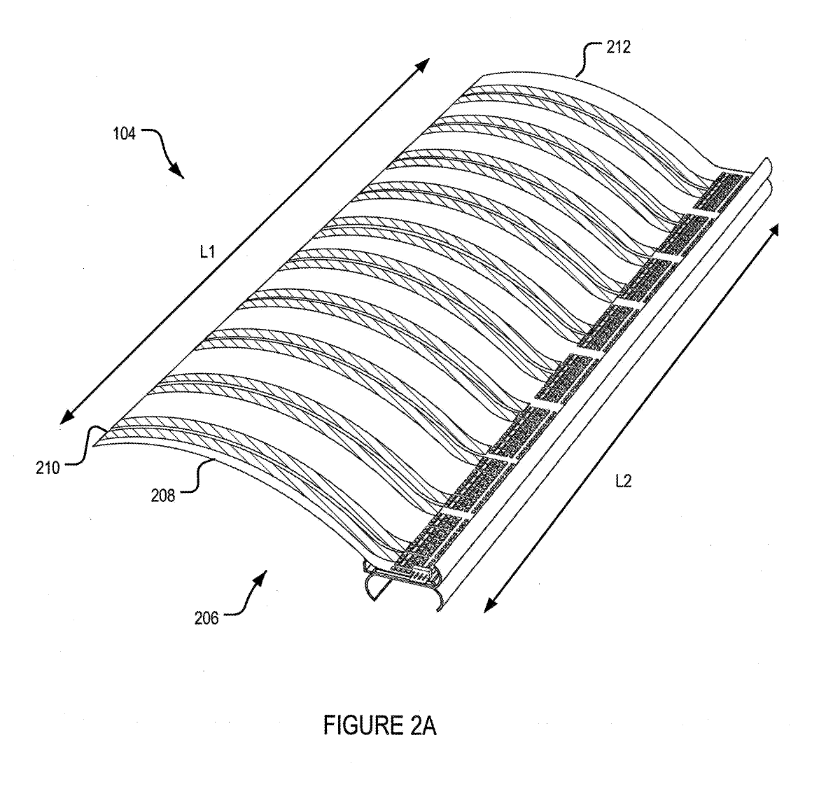

As shown in FIG. 2A, the flexible sheet 104 is rectangular in shape and includes a secured side 206 and three free sides 208, 210, 212. The secured side 206 and the free side 210 opposite to the secured side have substantially the same length (L1) as the length (L2) of the shaped member 108. In the present case, the width of the flexible sheet 104 is greater than the inner circumference of the tubular member 110 so that when the insert 200 (ref. FIG. 2C) is inserted into the tubular member 110 substantially all of an outer side surface 302 of the flexible sheet 104 makes contact with an inner wall of the tubular member 110 (ref. FIG. 1).

Referring now to FIGS. 2B and 2C, the flexible sheet 104 is formable around the outside of the shaped member 108 to form the insert 200 (ref. FIG. 2C) for insertion into the tubular member 110 (ref. FIG. 1). In the present case, forming the insert 200 entails wrapping the flexible sheet 104 from the secured side 206 around the outside of the shaped member 108 so that the flexible sheet 104 has an inner surface that wraps around the outside of the shaped member 108.

The insert 200 (ref. FIG. 2C) is deformable from an “initial configuration” to an “insertion” configuration in which its dimension is reduced for insertion into the tubular member 110 (ref. FIG. 1). The deformable nature of the insert 200 will be described subsequently.

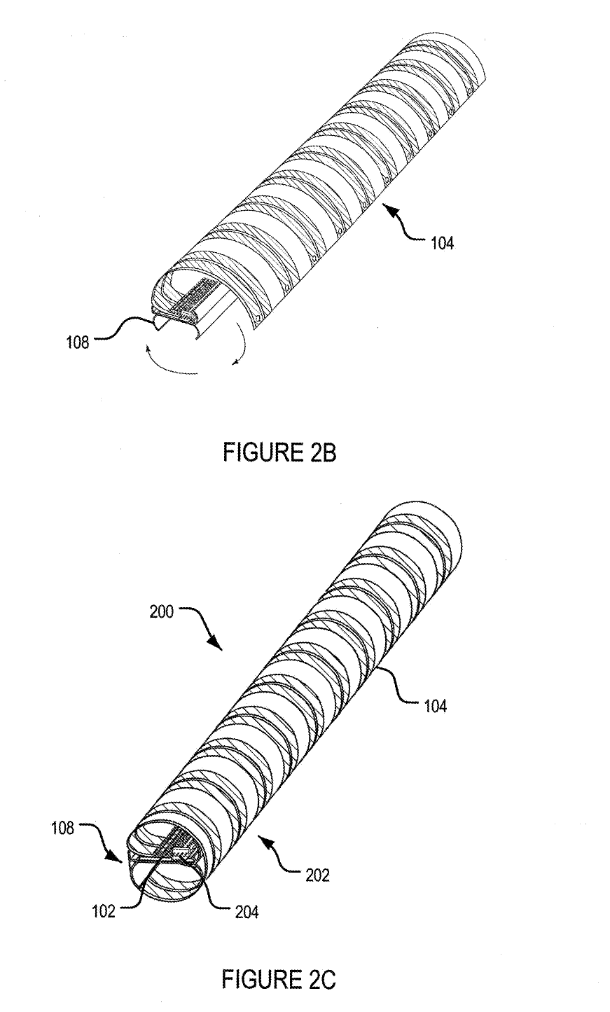

As is shown in FIG. 3A and FIG. 3B, the insert 200 is configured so that when it is located in the tubular member 110, an outer side surface 302 (ref. FIG. 3B) of the flexible sheet 104 (ref. FIG. 1), and thus the insert 200, bears against an inner wall 300 (ref. FIG. 3B) of the tubular member 110.

In the illustrated embodiment, the insert 200 has a generally tubular configuration that is sufficiently deformable to enable the insert 200 to be resiliently deformed from the “initial” configuration to the “insertion” configuration so that the outer side surface 302 of the flexible sheet 104, and thus the shape of the sensing elements 106-A, 106-B, substantially adopts the shape of an inner wall 300 of the tubular member 110. In the present case, the insert 200 adopts a generally cylindrical configuration but it will be appreciated that other configurations may be adopted.

Returning now to FIG. 2A, in the embodiment illustrated the electronics module 102 and the flexible sheet 104 are integrally formed as a single flexible printed circuit board (PCB) 202. However, it is to be appreciated that the electronics module 102 and the flexible sheet 104 need not be located on the same PCB 202, and thus could instead be formed as separate modules.

The sensing elements 106-A, 106-B (ref. FIG. 1) include flexible conductive strips on the PCB 202 that are arranged in pairs so as to form the plates of a capacitor. In the present case, the electronics module 102 is shown interconnected with each capacitor to form the sensing circuit. In the embodiment illustrated, the electronics module 102 includes multiple sensing circuits and so multiple pairs of conductive strips are provided. However, it will be appreciated that in other embodiments a single pair of conductive strips may be used, and thus a single sensing circuit provided. In the present case, the inclusion of multiple sensing circuits permits the sensor 50 to sense the soil moisture at multiple points along the length of the sensing unit 100. Thus, the flexible sheet 104 and the electronics module 102 are interconnected to form plural sensing circuits for sensing the soil moisture content of the soil medium surrounding the tubular member 110. Thus, when each pair of sensing elements 106-A, 106-B are connected to the electronics module 102, and the insert 200 is mounted in the tubular member 110, respective sensing circuits are formed that can then be used to sense the soil moisture content in the soil surrounding the tubular shaped member 108.

The specific configuration and operation of a suitable sensing circuit would be well within the knowledge of a skilled person.

Each sensing circuit is calibrated after manufacture to accurately account for any variations in the thickness of the tubular member 110 adjacent the various sensing elements 106-A, 106-B. As will be appreciated, a calibrated sensing unit 100 will be less susceptible to any errors in measurement due to variations in the properties of the tubular member 110 (ref. FIG. 1).

A connector 204 is provided to enable connection of the electronics module 102 on the PCB 202 to an external communications unit (not shown).

Although the previous description refers to the outer side surface 302 of the flexible sheet 104 as being located against the inner wall 300 of the tubular member 110, it will be appreciated by those skilled in the art that it is desirable to minimise any air gap between the outer side surface of any of the sensing elements 106-A, 106-B and the inner wall of the tubular member 110. It is recognised that the flexible sheet 104 may adopt many different configurations. This may result, for example, in the sensing elements 106-A, 106-B being embedded in a PCB so an outer side of a sensor element 106-A, 106-B may be in direct contact with the inner wall 300 of the tubular member or alternatively there may be a layer of material between the outer side of the sensing elements and the inner wall 300 of the tubular member 110. Alternatively, the flexible sheet 104 may adopt a form that does not include a flexible PCB. Irrespective of the arrangement/form of the flexible sheet and/or the sensing elements, it is preferred that any air gap between the inner wall 300 of the tubular member 110 and the outer side of the sensing elements 106-A, 106-B be minimised.

Continuing now with a description of the sensing elements 106-A, 106-B, the illustrated sensing elements 106-A, 106-B include a plurality of conductive strips arranged in pairs so as to form individual capacitors in a sensing circuit.

The capacitors are arranged in parallel with a fixed coil or inductor in the sensing circuit. In the present case, these components form part of an oscillator circuit that varies in frequency as the capacitance varies due to changes in the properties of the soil surrounding the tubular member 110 of the sensor 50.

A sensor 50 including a sensing unit 100 is advantageous because a sensing circuit including sensing elements 106-A, 106-B takes measurements only through the tubular member 110 and not through any significant air gap.

Accordingly, the measurements of the sensing elements 106-A, 106-B are not influenced to a significant extent by the existence of an air gap. The sensing elements 106-A, 106-B are thus much closer to the outer side surface 304 of the tubular member 110 and also closer to the soil whose properties are to be measured.

By minimising the air gap and calibrating the sensing unit 100 to account for variation in the properties (for example, the thickness) of the tubular member 110, more accurate measurements of the soil moisture content surrounding the tubular member 110 may be obtainable.

Due to the construction of the conductive strips forming the sensing elements 106-A, 106-B on the PCB 202, an electromagnetic field is established between the conductive strips and the soil medium on the outer side the tubular member 110. In the present case, the electromagnetic field is in the form of a substantially toroidal shaped electromagnetic field. The electromagnetic field is affected by both the relative permittivity of the soil and the moisture content of the soil medium.

As the relative permittivity of water is much higher than either air or soil, changes in soil moisture content will more strongly affect the permittivity and hence the capacity between the respective “driving” electrode 106-B and the “GND” electrode 106-A. This increase in capacitance causes the frequency of oscillation of the oscillator of the sensing circuit to change. The change in frequency of oscillation is subsequently interpreted by a micro-controller as a change in soil moisture content.

In the illustrated embodiment, the conductive strips are made from copper strips which are embedded in the flexible PCB 202 of the flexible sheet 104. As shown, the sensing elements 106-A, 106-B consist of a two types of conductive strips, namely a respective “driving” electrodes 106-B and “GND” electrodes 106-A that form the plates of a variable capacitor. In the present case, each of the GND electrodes 106-A are wider than the driving electrodes 106-B to provide a low inherent inductance.

The frequency of oscillation of the oscillator circuit of the sensing circuit on the PCB 202 is affected by the value of the dielectric constant of the soil medium, which in turn affects the capacitance between the “driving” electrode 106-B and the “GND” electrode 106-A. Each oscillator circuit includes a stabilising capacitor, in the form of “tank” capacitor (not shown), for maintaining the reactance of the sensing elements 106-A, 106-B as a capacitive reactance.

In the illustrated embodiment, each driving electrode 106-B interacts with adjacent GND electrodes 106-A to establish adjacent electromagnetic fields in the form of a pair of concentric toroidal fields. In other words, in one embodiment, each driving electrode 106-B interacts with two GND electrodes 106-A. It will of course be appreciated that it is not essential that each driving electrode 106-B interacts with two GND electrodes 106-A since in some embodiments each driving electrode 106-B will only interact with one GND electrode 106-A. However, an embodiment in which a driving electrode 106-B interacts with two GND electrodes 106-A may increase the effective size of the sensing area.

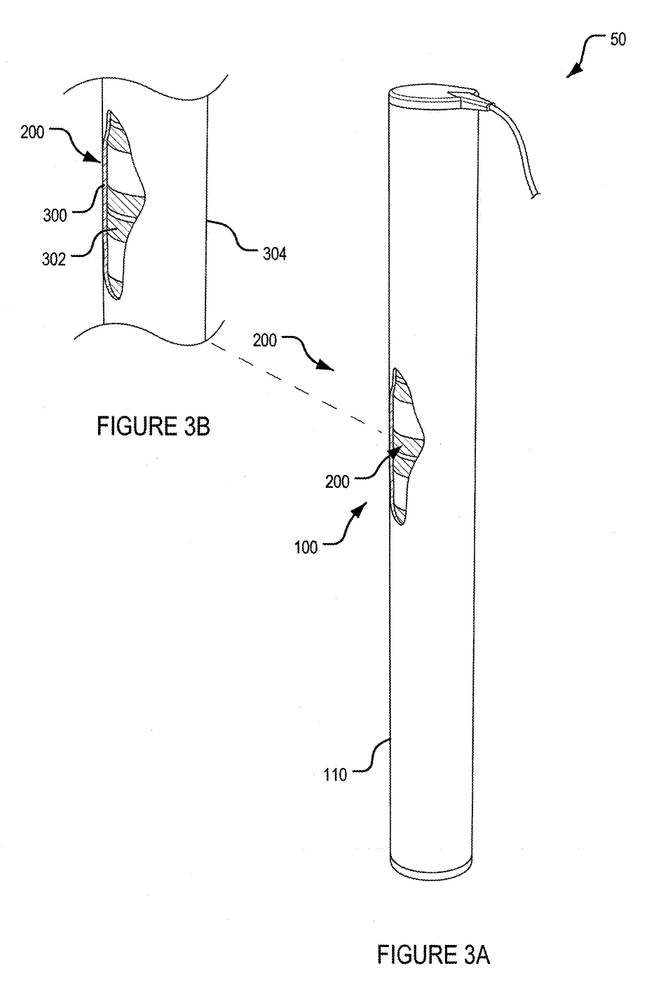

FIG. 4 shows a cross section of the insert 200 depicted in FIG. 2A and shows the shaped member 108 in more detail. The illustrated shaped member 108 includes resilient surfaces 400 that extend about a longitudinal axis of the shaped member 108. Each surface 400 is arranged to provide guides for shaping the flexible sheet 104 during forming the insert 200.

The guides shown here include outwardly projecting flanges 402, extending lengthwise along the shaped member 108, that are spaced apart with respect to one another to provide a spacing (shown as S1, S2, S3) therebetween.

In such an embodiment, after forming the insert 200 (ref. FIG. 2C), areas of the flexible sheet 104 spanning the spacings will be manipulable to an extent by applying a radially inward force thereto so as to reduce a dimension between the respective area of the flexible sheet 104 and the longitudinal axis of the shaped member 108. In the present case, the configuration of the flanges 402, combined with the resilient nature of the flexible sheet 104, biases the outer side surface 302 of the flexible sheet 104 against the inner wall 300 of the tubular member 110 after insertion of the insert 200 into the tubular member 110. Accordingly, the shape of the sensing elements 106-A, 106-B substantially adapts the shape of the inner wall 300 of the tubular member 110. In this manner the air gap is eliminated or at least substantially reduced.

Since, in the embodiment illustrated, the insert 200 is to adopt a generally tubular configuration, the flanges 402 are arcuate in shape to provide a guide that defines an inner dimension, such as an inner diameter, of the inner surface of the flexible sheet after formation of the insert 200.

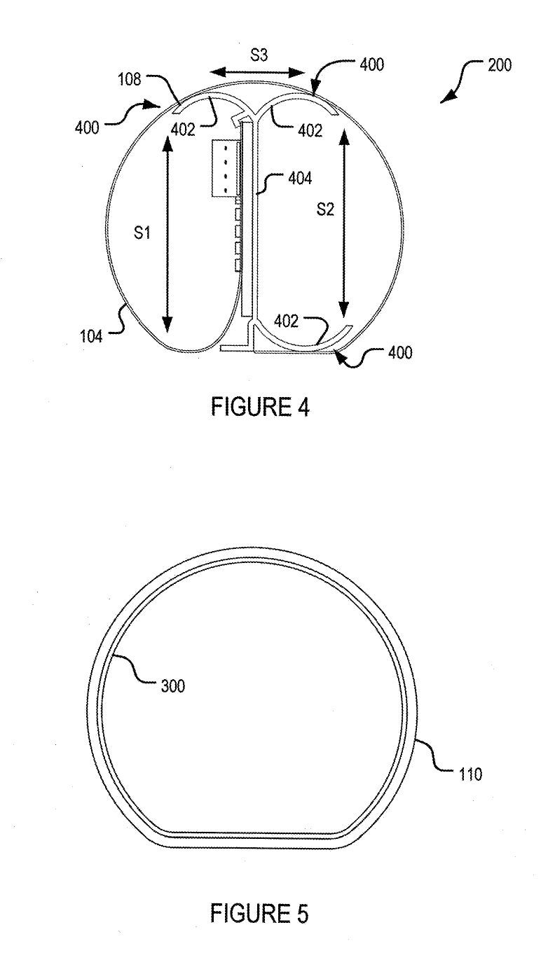

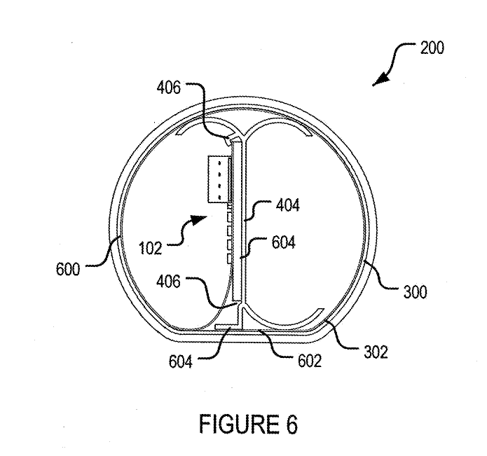

With reference now to FIG. 6, once inserted within the tubular member 110 the insert 200 will endeavour to return to its “initial” configuration. Subject to the tubular member 110 having an appropriate inner dimension, such as that depicted in FIG. 5, the resilient nature of the insert 200 will thus contribute to the outer side surface 302 of the flexible sheet 104 being biased against the inner wall 300 of the tubular member so that the shape of the one or more sensing elements 106-A, 106-B substantially adopts the shape of the inner wall 300 of the tubular member 110, thus substantially removing any air gap located therebetween.

The shaped member 108 depicted in FIG. 4 also includes a support arrangement for supporting the electronics module 102. In the present case, the support arrangement includes a planar section 404 for receiving and supporting the electronics module 102.

As shown in FIG. 6, the planar section 404 may include a web located between and connected to flanges 402 (ref. FIG. 4) of the shaped member 108. The illustrated web includes oppositely arranged clamping members 406 for securing the electronics module 102 to the web. In the present case, the web is resilient and sufficiently flexible to permit the position clamping members 406 to be manipulated with respect to each other for clamping and releasing the electronics module 102. In the present case, an adhesive strip 604, such as a double sided tape, is also used to secure the electronics module 102 to the web.

As is also shown in FIG. 6, the outer side surface 302 of the insert 200 and the inner wall 300 of the tubular member 110 are correspondingly shaped so as to resist rotation of the insert 200 about the longitudinal axis of the tubular member 110 when the insert 200 is inserted into the tubular member 110 (ref. FIG. 1). In the illustrated embodiment this is achieved by providing an outer side surface 302 that includes a curved surface 600 and a substantially flat surface 602. In the present case, the curved surface 600 has a truncated generally circular cross section along the length of the insert 200, and spans an azimuthal extent of more than 270 degrees. The flat surface 602 is formed in cooperation with a flat flange 604 forming part of the shaped member 108.

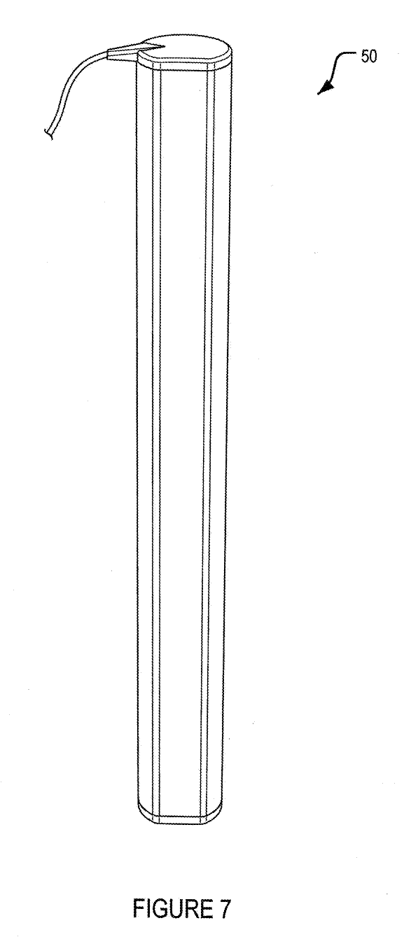

FIG. 7 shows a sensor 50 in the assembled configuration.

The embodiments have been described by way of example only and modifications within the spirit and scope of the invention are envisaged. Thus, it must be appreciated that there may be other various and modifications to the configurations described herein which are also within the scope of the present invention.

1. A sensing unit for sensing a parameter of a medium surrounding a tubular member, the sensing unit including:
an electronics module;
a flexible sheet including one or more sensing elements interfacing with the electronics module to form a sensing circuit for sensing the parameter of the medium; and
a shaped member;
wherein the flexible sheet is formable around the outside of the shaped member to provide an insert for insertion into the tubular member, and wherein after insertion into the tubular member the insert bears against an inner wall of the tubular member so that the shape of the one or more sensing elements adapts to the shape of the inner wall of the tubular member.

2. A sensing unit according to claim 1 wherein the medium is a soil medium, and wherein the sensed parameter is soil moisture.

3. A sensing unit according to claim 1 wherein the flexible sheet is a flexible printed circuit board (PCB) arranged in use for connection to the electronics module and wherein the one or more sensing elements are formed integrally with the PCB.

4. A sensing unit according to claim 3 wherein the electronics module is located on the flexible PCB.

5. A sensing unit according to claim 1 wherein the one or more sensing elements include flexible conductive strips.

6. A sensing element according to claim 5 wherein the flexible conductive strips are arranged in pairs, and wherein each conductive strip of a pair forms a capacitive element in the sensing circuit.

7. A sensing unit according to claim 6 wherein an outer side surface of the insert and an inner wall of the tubular member are correspondingly shaped for resisting rotation of the insert relative to the tubular member when the insert is inserted into the tubular member.

8. A sensing unit according to claim 7 wherein the outer side surface of the insert includes a curved surface extending between a substantially flat surface.

9. A sensing unit according to claim 8 wherein the curved surface has a truncated generally circular cross section along the length of the insert, and spans an azimuthal extent of at least 270 degrees.

10. A sensing unit according to claim 8 wherein the substantially flat surface is aligned, after insertion, with a flat surface of the inner wall of the tubular member to restrict rotation of the insert.

11. A sensing unit according to claim 1 wherein the insert adopts a generally tubular configuration that is sufficiently deformable to enable the insert to be resiliently deformed from an “initial” configuration to an “insertion” configuration.

12. A sensing unit according to claim 1 wherein the flexible sheet is rectangular in shape.

13. A sensing unit according to claim 12 wherein the rectangular shape includes a secured side and three free sides, and wherein the secured side has substantially the same length as the length of the shaped member and wherein the width of the rectangular sheet is substantially the same as, or greater than, an inner circumference of the tubular member.

14. A sensing unit according to claim 13 wherein the insert is formed by wrapping the flexible sheet from the secured side and around the shaped member so that the flexible sheet has an inner side surface that wraps around the shaped member.

15. A sensing unit according to claim 1 wherein the shaped member includes one or more resilient surfaces for contacting areas of an inner side surface of the flexible sheet when the flexible sheet is formed around the outside of the shaped member.

16. A sensing unit according to claim 1 wherein the shaped member includes a web, the web including oppositely arranged clamping members for receiving the electronics module, wherein the web is resilient to permit the relative position of clamping members to be manipulated with respect to each other for clamping and releasing the electronics module, so that when the shaped member is inserted in the tubular member the clamping members are biased to secure the electronics module therebetween.

17. A sensing unit according to claim 16 wherein after forming the insert, the flexible sheet surrounds the web so that the electronics module is located within an area surrounded by an inner surface of the flexible sheet.

18. A sensing unit according to claim 1 wherein the electronics module is located along an edge of the flexible sheet.

19. A sensor for sensing moisture content of a soil medium, the sensor including:
a tubular member;
an electronics module;
a flexible sheet including one or more sensing elements interfacing with the electronics module to form a sensing circuit for sensing the parameter of the soil medium surrounding the tubular member; and
a shaped member;
wherein the flexible sheet is formed around the outside of the shaped member to provide an insert which is located within the tubular member and wherein the one or more sensing elements substantially adopts the shape of an inner wall of the tubular member.

20. A sensor according to claim 19 wherein after insertion into the tubular member the insert bears against an inner wall of the tubular member so that the one or more sensing elements substantially adopts the shape of the inner wall of the tubular member.

21. A sensor according to claim 20 wherein substantially all of the outer side surface of the flexible sheet makes contact with the inner wall of the tubular member.

22. A sensing unit for sensing a parameter of a medium, the sensing unit including:
an electronics module;
a flexible sheet including one or more sensing elements interfacing with the electronics module to form a sensing circuit for sensing the parameter of the medium surrounding a tubular member; and
a shaped member;
wherein the flexible sheet is formable around the outside of the shaped member to provide an insert for insertion into the tubular member.

23. An insert for inserting into a tubular member of a sensing unit for sensing a parameter of a medium, the insert including:
a flexible sheet including one or more sensing elements for interfacing with an electronics module to form a sensing circuit for sensing the parameter of the medium surrounding the tubular member; and
a shaped member;
wherein the insert is formed by forming the flexible sheet around the outside of the shaped member.

24. (canceled)

25. (canceled)
